(19) United States
(12) Patent Application Publication
(10) Pub. No.: US 2022/0406465 A1
(43) Pub. Date: Dec. 22, 2022

(54) MENTAL HEALTH RISK DETECTION USING GLUCOMETER DATA

(71) Applicant: TELADOC HEALTH, INC., Purchase, NY (US)

(72) Inventors: Jessica Yu, San Ramon, CA (US); Carter Chiu, Sunnyvale, CA (US); Yajuan Wang, Palo Alto, CA (US); Eldin Dzubur, Sunnyvale, CA (US); Wei Lu, Mountain View, CA (US); Julia Hoffman, San Jose, CA (US)

(21) Appl. No.: 17/590,384

(22) Filed: Feb. 1, 2022

Related U.S. Application Data
(60) Provisional application No. 63/144,364, filed on Feb. 1, 2021.

(51) Int. Cl. G16H 50/30

(52) U.S. Cl. CPC G16H 50/30

(57) ABSTRACT

A system, method, and computer program product for passively informed mental health risk prediction. The system may include receiving mental health risk input signals from a blood glucometer and other devices. The mental health risk signals may include glucometer data, demographic data, and other data. The glucometer data for the subject may include at least one blood glucose value. The mental health risk input signals are input into a machine learning system. The machine learning system has been previously trained with mental health risk input signals and mental health status data for a plurality of subjects. The machine learning system outputs a prediction of mental health risk for the subject. The machine learning system may comprise a neural network.

### CROSS-REFERENCE TO RELATED APPLICATIONS

This application claims the benefit of U.S. Provisional Application No. 63/144,364, Feb. 1, 2021, for MENTAL HEALTH RISK DETECTION USING GLUCOMETER DATA, which is hereby incorporated by reference in its entirety.

### TECHNICAL FIELD

The present disclosure relates generally to telehealth systems and more specifically to detecting mental health risk in diabetes patients.

### BACKGROUND

More than 10% of the United States population—at least 34.2 million people—is affected by diabetes mellitus (hereinafter “diabetes”). The mental health needs among people with diabetes are well documented. Approximately 25% of people with diabetes experience significant depressive symptoms and 10% to 15% are formally diagnosed with a depressive disorder. Approximately 27% to 40% of people with diabetes experience significant anxious symptoms and 14% are formally diagnosed with generalized anxiety disorder (GAD). These statistics are up to 2 to 3 times greater than for people without diabetes, and do not include the increased prevalence of other mental health concerns such as disordered eating and diabetes distress. Beyond prevalence, the health and economic burdens associated with co-morbid diabetes and mental health are significant. Those with co-morbid diabetes and mental health have been found to be less adherent to diabetes treatment recommendations, including diet, exercise, medication use, glucose monitoring, and medical appointments, and at greater risk for adverse medical outcomes. Healthcare costs for those with co-morbid diabetes and mental health have been estimated to be $4 billion to $9 billion greater than for those without.

Proactive detection of mental health needs among people with diabetes could facilitate early intervention, thereby improving the overall health and quality of life of people with diabetes and reducing the health and economic burdens placed on the diabetes population and healthcare system as a whole. Yet, the current state of mental health detection in people with diabetes is poor. Despite recommendations by the American Diabetes Association and United States Preventive Services Task Force to routinely evaluate people with diabetes for mental health needs, only 25% to 50% of people with diabetes who have depression receive a mental health diagnosis and intervention. There are myriad reasons for this, including a shortage of mental health professionals available to offer assessment and intervention, lack of mental health knowledge among primary care providers who most often care for subjects with diabetes, and limited access to mental health screening tools in healthcare practices offering services to subjects with diabetes.

### SUMMARY

There is a clear need for improvement in the methods used to detect mental health needs in people with diabetes. Existing methods are not scalable in that they rely on trained professionals to deliver assessment and are effortful and time-intensive in that they require subjects to actively respond to questions about their emotional and mental state. Newer methods, such as passive sensing and ecological momentary assessment (EMA), are more scalable and less effortful and time-intensive.

Passive sensing refers to the capture of data about a person without extra effort on their part. EMA refers to the repeated sampling of behavior in real-time within the natural environment. Both methods can be integrated into or with devices and services people with diabetes already utilize in their daily lives, such as blood glucose (BG) meters, smartphones, and health coaching, and therefore require little to no additional effort and time. And because they enable the collection and processing of data in real time and context, they can serve as a springboard for just-in-time adaptive interventions—interventions that can be used for people at the very moment they need them.

Passive sensing and EMA have previously been examined in the detection of mental health concerns among the general population, but few have focused on the detection of mental health needs among people with diabetes in particular. Previous efforts focused narrowly on smartphones as data warehouses, relying on accelerometer, GPS, ambient light sensor, and call log data. They have also used traditional assessments and measures as validation measures—for example, the Patient Health Questionnaire-9 (PHQ-9). No known effort to date has attempted to detect mental health concerns in people with diabetes via BG meters. However, there is a rationale for doing so. People with diabetes are encouraged to engage with their BG meters at regular intervals, BG monitoring has been correlated with psychological effects, and BG engagement and BG level may indicate mood or stress. Further, BG meter data can be paired with data from other sources for a robust view of a person's behavioral and emotional profile.

A computer-implemented method for mental health risk prediction may include receiving mental health risk input signals, which may include glucometer data and demographic data. The glucometer data for the subject may include at least one blood glucose value. The mental health risk input signals are input into a machine learning system. The machine learning system has been previously trained with mental health risk input signals and mental health status data for a plurality of subjects. The machine learning system outputs a prediction of mental health risk for the subject. The machine learning system may comprise a neural network.

The glucometer data may include one or more of the following: a total number of blood glucose checks performed within a particular time interval; a proportion of days with blood glucose checks; a minimum blood glucose value; a mean blood glucose; a maximum blood glucose value; a standard deviation of blood glucose values; a proportion of blood glucose values below 70 mg/dl; proportion of blood glucose values above 180 mg/dl; a proportion of blood glucose checks with wellness indicated; a proportion of blood glucose checks with unwell state indicated; a proportion of blood glucose checks without a reported feeling tag; and a proportion of blood glucose checks with exercise indicated.

The demographic data may include one or more of the following: age; body mass index (BMI); gender; race; diabetes status; and smoking status.

The mental health status data may include one or more of the following: mental health medications prescribed for one or more of the plurality of subjects; mental health assessments made for one or more of the plurality of subjects; mental health insurance claims reported for one or more of the plurality of subjects; and mental health interventions provided for one or more of the plurality of subjects.

The method may include the following initial steps: for each subject of a first set of subjects, collecting mental health risk input signals including: glucometer data including a glucose value, demographic data, and mental health status data; creating a training set comprising the glucometer data, demographic data, and mental health status data for each subject of the first set of subjects; and training the machine learning system in a training stage using the training set to create an machine learning model.

The method may further include the following steps: for each subject of a second set of subjects, collecting mental health risk input signals including: glucometer data including a glucose value, demographic data, and mental health status data; creating a validation set comprising the glucometer data, demographic data, and mental health status data for each subject of the second set of subjects; validating the ML model in a validation stage using the validation set; and updating the ML model in response to one or more validation errors.

The mental health risk input signals may further include coaching data relating to contacts between the subject and a coach. The coaching data may include one or more of the following: a number of coaching alerts triggered; a number of successful coach-subject contacts; a number of successful coach-subject contacts by phone; a number of attempted unsuccessful coach-subject contacts by phone; a number of successful coach-subject contacts by text; a number of attempted coach-subject unsuccessful contacts by text; a number of successful coach-subject contacts by email; a number of attempted unsuccessful coach-subject contacts by email; a number of successful coach-subject contacts by glucometer; a number of attempted unsuccessful contacts by glucometer; a number of coaching sessions where subjects took scheduled a future coaching session; and average minutes spent on a coaching alert interaction.

The mental health risk input signals may further include event data relating to one or more of frequency, duration, interactivity, and consistency of interaction sessions associated with use by the subject of a mobile application or web portal.

Another aspect of the disclosure is a system for mental health risk prediction. The system may include an input device for receiving mental health risk input signals for a subject. The mental health risk input signals may include glucometer data and demographic data for the subject. The glucometer data may include at least one blood glucose value. The system may include a machine learning system for receiving the mental health risk input signals. The machine learning system may be previously trained with mental health risk input signals and mental health status data for a plurality of subjects. The system may further include an output device for providing a prediction of mental health risk for the subject output by the machine learning system. The machine learning system may comprise a neural network.

The glucometer data may include one or more of the following: a total number of blood glucose checks performed within a particular time interval; a proportion of days with blood glucose checks; a minimum blood glucose value; a mean blood glucose; a maximum blood glucose value; a standard deviation of blood glucose values; a proportion of blood glucose values below 70 mg/dl; proportion of blood glucose values above 180 mg/dl; a proportion of blood glucose checks with wellness indicated; a proportion of blood glucose checks with unwell state indicated; a proportion of blood glucose checks without a reported feeling tag; and a proportion of blood glucose checks with exercise indicated.

The demographic data may include one or more of the following: age; body mass index (BMI); gender; race; diabetes status; and smoking status.

The mental health status data may include one or more of the following: mental health medications prescribed for one or more of the plurality of subjects; mental health assessments made for one or more of the plurality of subjects; mental health insurance claims reported for one or more of the plurality of subjects; and mental health interventions provided for one or more of the plurality of subjects.

The input device, for each subject of a first set of subjects, may collect mental health risk input signals. The mental health risk input signals may include: glucometer data including a glucose value, demographic data, and mental health status data. The machine learning system may initially receive a training set comprising the glucometer data, demographic data, and mental health status data for each subject of the first set of subjects to create an ML model.

The input device, for each subject of a second set of subjects, may collect mental health risk input signals. The mental health risk input signals may include: glucometer data including a glucose value; demographic data; and mental health status data. The machine learning system may validate, during a validation stage, the machine learning model using a validation set and update the machine learning model. The validation set may comprise glucometer data, demographic data, and mental health status data for each subject of the second set of subjects.

The mental health risk input signals may further include coaching data relating to contacts between the subject and a coach. The coaching data may include one or more of the following: a number of coaching alerts triggered; a number of successful coach-subject contacts; a number of successful coach-subject contacts by phone; a number of attempted unsuccessful coach-subject contacts by phone; a number of successful coach-subject contacts by text; a number of attempted coach-subject unsuccessful contacts by text; a number of successful coach-subject contacts by email; a number of attempted unsuccessful coach-subject contacts by email; a number of successful coach-subject contacts by glucometer; a number of attempted unsuccessful contacts by glucometer; a number of coaching sessions where subjects took scheduled a future coaching session; and average minutes spent on a coaching alert interaction.

The mental health risk signals may further include event data relating to one or more of frequency, duration, interactivity, and consistency of interaction sessions associated with use by the subject of a mobile application or web portal.

### BRIEF DESCRIPTION OF THE DRAWINGS

In order to describe the manner in which the above-recited and other advantages and features of the disclosure can be obtained, a more particular description of the principles briefly described above will be rendered by reference to specific embodiments thereof which are illustrated in the appended drawings. Understanding that these drawings depict only example embodiments of the disclosure and are not therefore to be considered to be limiting of its scope, the principles herein are described and explained with additional specificity and detail through the use of the accompanying drawings in which:

FIG. 1 is a diagram of a system for training a machine learning model for mental health risk detection in diabetes patients according to one embodiment of the present disclosure.

FIG. 2 shows examples of mental health risk input signals relating to demographics data and glucometer data in a system for detecting mental health risk in diabetes patients.

FIG. 3 shows examples of mental heath risk input signals relating to coaching data and event data in a system for detecting mental health risk in diabetes patients.

FIG. 4 is a diagram of a system for detecting mental health risk in diabetes patients according to one embodiment of the present disclosure.

FIG. 5 illustrates a process of detecting mental health risk in diabetes patients according to one embodiment of the present disclosure.

FIG. 6 depicts an example computing system that may implement various systems and methods according to embodiments of the present disclosure.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without departing from the spirit and scope of the disclosure. Moreover, although the present disclosure discusses the detection of mental health risk in diabetes patients, a person skilled in the art will recognize that the systems and methods described herein can be applied to detect mental health risk in patients with other medical conditions whose management involves regular interaction with devices, mobile apps, web portals, coaches, nurses, and/or other healthcare providers.

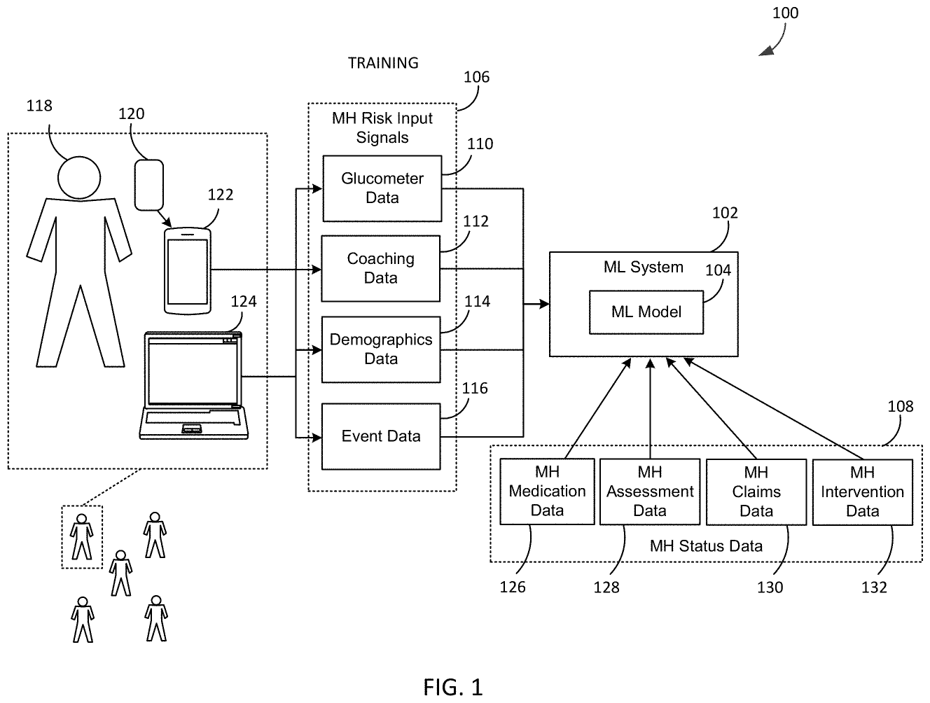

FIG. 1 illustrates a system 100 for training a machine learning (ML) system 102 for mental health (MH) risk detection in diabetes patients. The ML system includes a ML model 104 that may be trained using a plurality of MH risk input signals 106 and MH status data 108 from a plurality of subjects.

The MH risk signals 106 may generally be divided into four categories based on their respective sources: glucometer data 110; coaching data 112; demographics data 114; and event data 116. These data may be collected from a plurality of subjects, who may be participating in a diabetes management program. An example of such a program is that provided by Livongo Health, Inc., of Mountain View, Calif.

Each subject 118 may have a blood glucometer 120, such as the Livongo BG300. Generally, the glucometer 120 is used to analyze a sample of the subject's blood and calculate the subject's blood glucose level. The glucometer 120 may store a history of the subject's glucose checks in database in an on-board data storage system. Each glucose check record may be stored in association with the date and time of the check, as well as other information. For example, when the subject 118 uses the glucometer 120 to check their blood glucose level, the glucometer may query the subject 118 for additional information. The glucometer may prompt the subject 118 to input how they are feeling at the time of the check; whether they consumed a meal before the check; and/or whether the subject exercised before the check. All or portions of this glucometer data 110 may be included among the MH risk input signals provided to the ML system. By way of example, the glucometer data 110 may be transferred via a wireless Bluetooth connection to another device such as the subject's smartphone 122.

The subject's smartphone 122 and/or other web-enabled device, such as a laptop 124, may also be sources of MH risk input signals 106. By way of example, the subject 118 may use their laptop 124 to enroll in the diabetes management program and input demographics data 114 when establishing their patient profile. In another example, the subject may log into a web portal and interact with a diabetes coach via laptop 124. In yet another example, the subject may interact with coaches via a mobile app installed on the smartphone 122. And in yet another example, the subject may use the mobile app on their smartphone 122 to share health progress reports with friends and family members via one or more social media platforms. Subjects may also use the mobile app or web portal for food logging and integration with other devices and programs, such a blood pressure device and hypertension management program, and connection to a Fitbit® device and/or Apple® Healthkit®.

The ML system 102 may employ any suitable machine learning approach. In one embodiment, the ML system 102 employs a supervised machine learning system such as a deep learning neural network or gradient boosting algorithm such as LightGBM. The ML system 102 may be embodied in a software module that executes on one or more servers coupled to one or more networks (not shown) that connect other devices in system 100, such as the Internet. Alternatively, the ML system 102, or portions of it, may execute on the subject's devices or a healthcare provider's device.

To train the ML model 104 to detect MH risk, the ML system 102 may be supplied with a training set including MH risk signals 106 for a plurality of subjects and associated MH status data 108 for the same subjects. In one embodiment, the training set provided to the ML system 102 includes a set of MH risk input signals for a plurality of subjects over a predetermined period of time. Each subject whose data is included in the training set may also be labelled to indicate whether the subject is known to have a MH condition. This data labelling may be accomplished using the MH status data 108. The MH status data 108 may come from various sources, including MH medication data 126, MH assessment data 128, MH claims data 130, and MH intervention data 132. The system 100 may query and receive this information from a subject's pharmacy, medical record, medical provider, insurance provider, diabetes management program profile, and/or other sources of health information on the subject 118.

The MH medication data 126 may include information on whether each subject has been prescribed and/or filled a prescription for certain medications used to treat MH conditions. For example, if the MH medication data 126 indicates that a particular subject has received and filled a prescription for Prozac or its generic equivalent, that particular subject may be labelled as having a MH condition in the training set.

The MH assessment data 128 may include information from past medical assessments of the subjects. For example, if the MH assessment data 128 indicates that a particular subject was seen by a psychiatrist and diagnosed with depression, that particular subject may be labelled has having a MH condition in the training set. Similarly, the MH intervention data 128 may include information on past MH-related interventions for the subjects. For example, if the MH intervention data 128 indicates that a particular subject received suicide counseling, that particular subject may be labelled has having a MH condition in the training set.

The MH claims data 130 may include information related to MH medication, MH assessment, and MH intervention, like those discussed above, but derived from each subject's insurance claims information, as opposed to pharmacy records, medical records, or other sources.

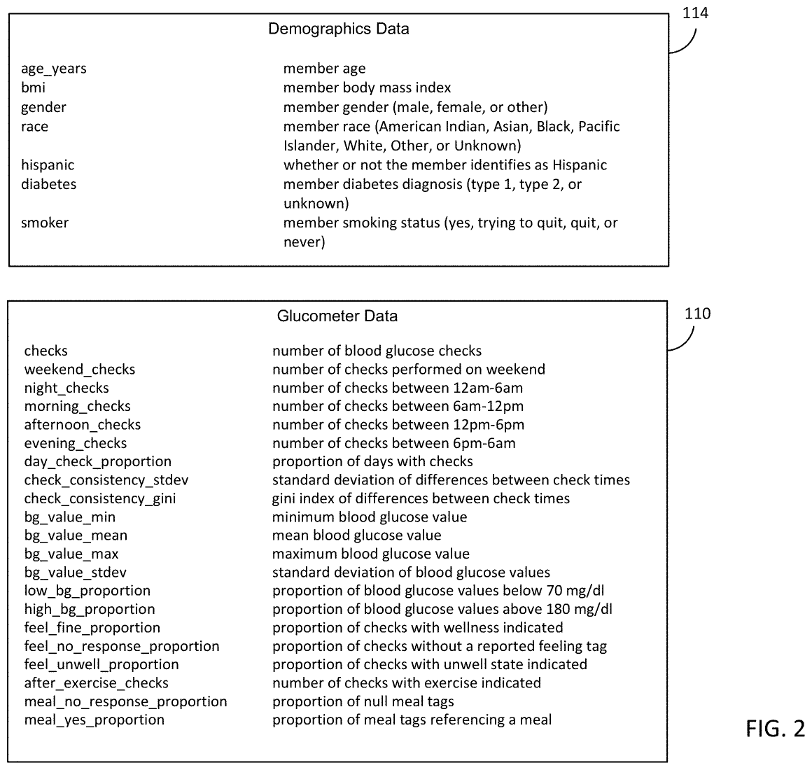

FIG. 2 defines a number of MH risk input signals in the demographics data 114 and glucometer data 110 categories discussed above with reference to FIG. 1. Demographic factors such as age, gender, ethnicity, and race have been shown to be related to mental health. Thus, these and other factors may be included among the MH risk input signals to the ML system 102.

Glucometer data 110 in FIG. 2 defines a number of signals in the glucometer data 110 category. The glucometer is a frequent interaction point for people with diabetes, and particularly those enrolled in diabetes management programs. Low rates of blood glucose monitoring and poorer blood glucose control have been linked to depression among those with diabetes. Similarly, depression, anxiety, and stress symptoms are greater among people with diabetes than people without. Differences in glucometer usage is particularly informative of conditions such as depression when examining usage time of day and weekday. Accordingly, volume of BG checks, BG values and variations, and responses to questions to assess context such as current emotional state and check time of day and day of week, and other signals in the glucometer data 110 may be useful indicators in assessing MH risk in a subject.

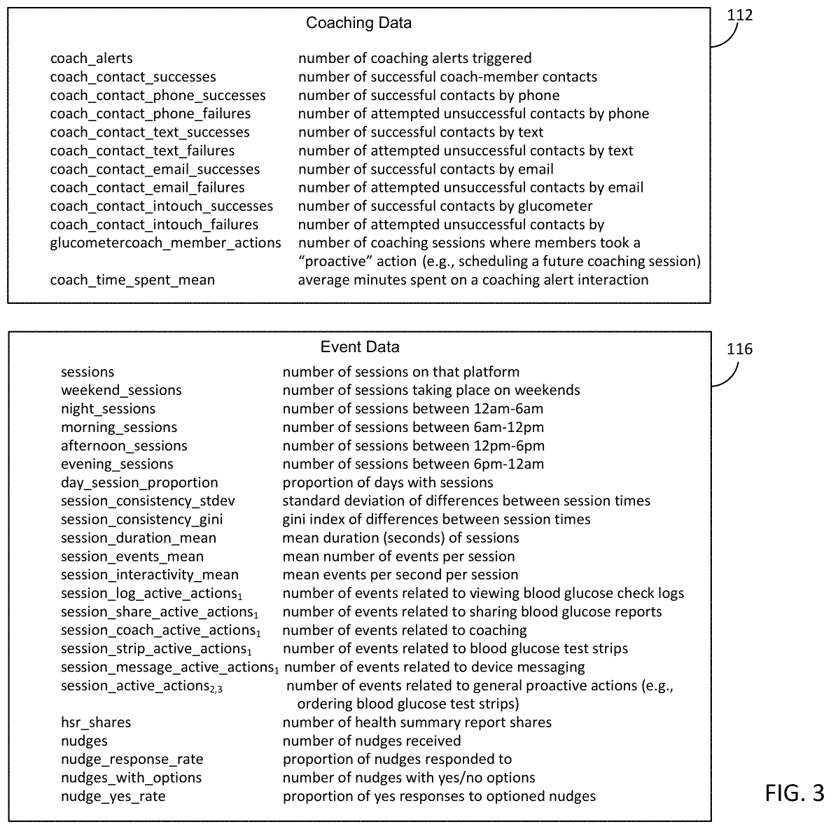

FIG. 3 defines a number of MH risk input signals in the coaching data 112 and event data 116 categories. With regard to the coaching data 112, in some diabetes management programs, coaches may contact participants under certain conditions. As numerous studies have affirmed, there are relationships between sociability and mental health. For example, fewer calls and fewer incoming SMS (or “text”) messages have been linked to depression. In addition, frequency and duration of conversations have been shown to be useful in evaluation of bipolar disorder. The coaching data 112 input signals shown in FIG. 2 can serve as a poxy for sociability. The number, rate, and/or changes in rates of successful or failed coaching contacts and time spent interacting with coaches can provide meaningful insight into a subjects MH risk. Further, changes in the number or rate of successful or unsuccessful contacts via specific communication platforms (e.g., voice calling, text messaging, email, and/or glucometer-based communication) can also be used to identify MH risk in a subject.

The event data 116 shown in FIG. 3 may also provide useful MH risk input signals. The event data 116 may include data concerning all types of interactions between a subject and the diabetes management program across all available platforms, including mobile apps and web platforms associated with the program. By way of example, the system may collect the frequency, duration, interactivity, and consistency of interaction sessions with the diabetes management program mobile app and web portal. In addition, the system may track whether, how often, and how consistently subjects voluntarily share reports with friends and family members, as well as interactions with pop-up reminders.

To the extent that the data received from the various inputs (e.g., glucometer, mobile device, laptop, and demographics) are not already formatted or expressed in the form or definition of the MH risk input signals 106 discussed above, the ML system 102 may include a pre-processor for formatting the data accordingly. Alternatively, this step can occur elsewhere in the system. For example, if the received glucometer data 110 includes a series of blood glucose values, but not a standard deviation of blood glucose values, the ML system 102 or some other processor may calculate the standard deviation of the blood glucose values and either insert the standard deviation into the MH risk input signals or otherwise use the calculated standard deviation as the corresponding MH risk input signal 106.

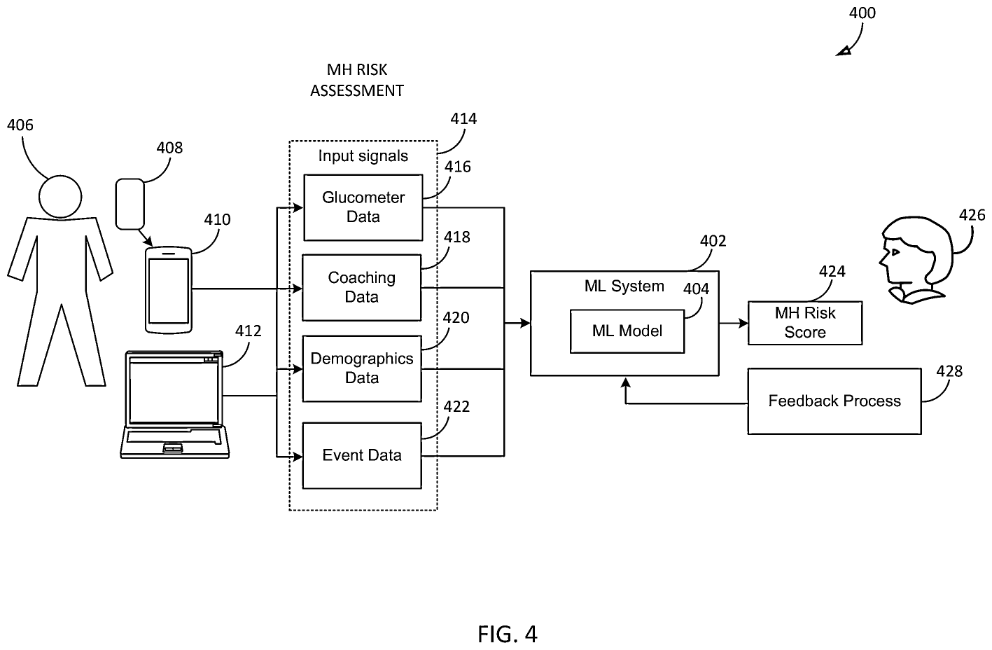

FIG. 4 shows a system 400 for mental health risk assessment in diabetes patients in accordance with the present disclosure. The system 400 may include an ML system 402 with an ML model 404 that has been trained using a training set of mental health risk input signals and mental health status data as discussed above with respect to FIGS. 1-3. A diabetes patient or subject 406 may interact with various devices as part of a diabetes management program. For example, the subject 406 may check their blood glucose levels using a glucometer 408. The subject may also interact with a diabetes coach using a mobile app on their smartphone 410 or by logging on to a web portal using a web-enabled device such as a laptop 412. Data about these interactions may be collected by the system in the form of MH risk input signals 414. These signals may include glucometer data 416, coaching data 418, demographics data 420, and event data 422, as discussed above.

The collected MH risk input signals may be fed into the ML system 402 for analysis using the trained ML model 404, which then outputs an MH risk score 424. The MH risk score may indicate a degree of confidence that the subject 406 presents with a mental health condition. The MH risk score 424 may be output as a statistical confidence value expressed as, for example, a decimal between 0 and 1 or a percentage between 0 and 100. In another embodiment, the MH risk score 424 may presented as a confidence value that has been discretized into a number of ranges, such as “low risk”, “medium risk”, and “high risk”. The MH risk score 424 may additionally or alternatively be presented graphically, such as with a bar graph, gauge, dial, or color scheme. The MH risk score 424 may be presented on a display interface to a healthcare provider 426. In one embodiment, the score 424 could be made available to or otherwise presented to a coach or healthcare provider during a communication session with the subject 406. The MH risk score 424 may also serve as an input to another computer program or application, or otherwise be used to automatically trigger an alert or notification to a healthcare provider 426 that the subject 406 presents a mental health risk.

The ML system 402 may also include a feedback process 428 to update and refine the ML model 404. The feedback can come from any number of sources, including the subject's coaches, healthcare providers, family, friends, the subject themselves, and/or additional or other training data. In one embodiment, the system 400 may determine, based on the subject's input signals 414 that the subject 406 has a high MH risk score. The system 400 may alert a healthcare provider 426 that the subject 406 has a high MH risk score. The healthcare provider 426 may than schedule an evaluation with the subject 406. Once the subject has been evaluated, the healthcare provider 426 may interface with the feedback process 428 to indicate whether the evaluation revealed the presence of an MH condition in the subject 406. The healthcare provider's evaluation may also be automatically fed back into the ML system 402 via the feedback process 428. The ML system may then update its internal model(s) 404 and provide different weights to various inputs, thereby improving the accuracy of the MH risk score 424 in the future.

The feedback process 428 may update the machine learning system 402, using, for example, a gradient descent algorithm and back propagation and the like as will be apparent to a person having ordinary skill in the art. In some examples, the machine learning system 402 may be updated in real time or near real time. In other examples, the machine learning system 402 may perform model updates as a background process on a mirror version of the machine learning system 402 and directly update the machine learning system 402 once the mirror version has converged on an updated model. In still other examples, the feedback process 428 may perform updates on a schedule or through a batch process. The updates can be performed on a singular device or may be performed across parallelized threads and processes and the like.

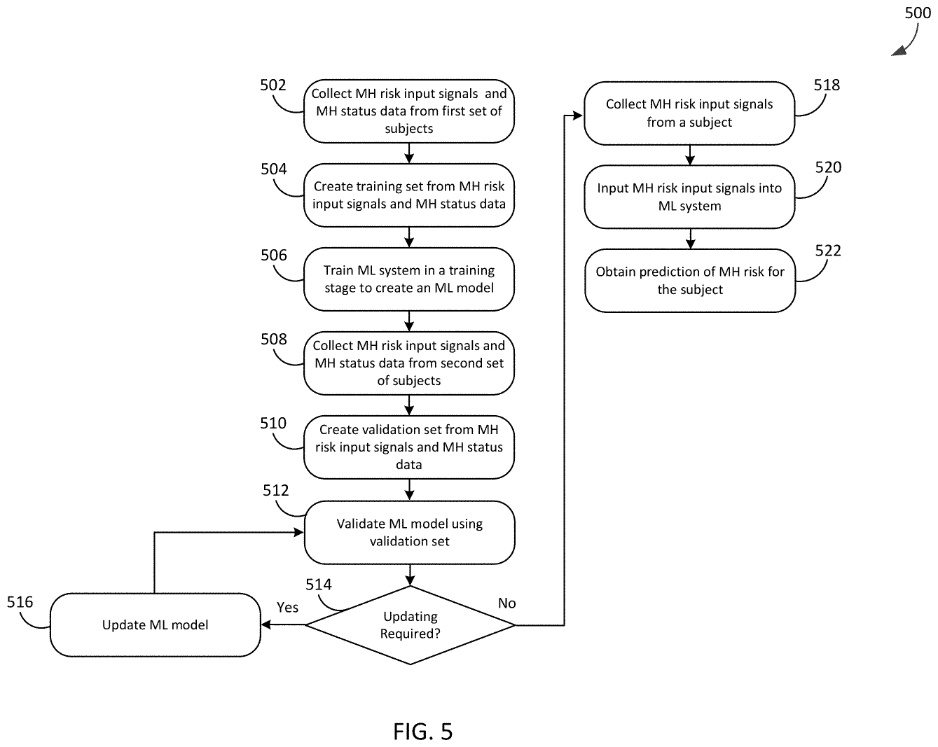

FIG. 5 is a flowchart illustrating a process 500 for detecting MH risk in diabetes patients using blood glucometer data in accordance with the present disclosure. The process may begin at step 502 with the collection of MH risk input signals and MH status data from a first set of subjects. Next, at step 504, a training set is created using the collected MH risk input signals and MH status data. In one embodiment, this involves using the MH status data to label each subject's associated input signals as either having or not having a mental health condition. At step 506, the training set is provided to the ML system to in a training stage to create the ML model.

Next, the ML model created in step 506 may be validated. In one embodiment, this is accomplished by collecting MH risk input signals and MH status data from a second set of subjects at step 508 and creating a validation set from those data at step 510. The validation set is then provided to the ML system and the results are validated at step 512. At step 514, it is determined whether the model requires updating. If so, the model is updated at step 516.

After validation, the process may proceed to step 518, where the model may be deployed within a system such as that described above with respect to FIG. 4 and used to predict MH risk in diabetes patients. At step 518, MH risk input signals are collected from a subject. At step 520, the MH risk input signals are provided to the trained and validated ML system. At step 522, the ML system produces an MH risk prediction score for the subject, which may be presented to a healthcare provider, stored in a database, or otherwise used to alert or notify a healthcare provider of the subject's MH risk. In one embodiment, if the MH risk score exceeds a predetermined threshold (e.g., a confidence score above 0.5), the system may send an electronic message to a healthcare provider alerting them of the subject's MH risk.

In one embodiment, the ML model consists of an ensemble of constituent models trained on random subsets of the training data. Each model may be tuned using any suitable method, such as automated hyperparameter tuning using the hyperopt Python library with 5-fold cross-validation on the training set. The outputs of each of the constituent models may be averaged to obtain a single MH risk score.

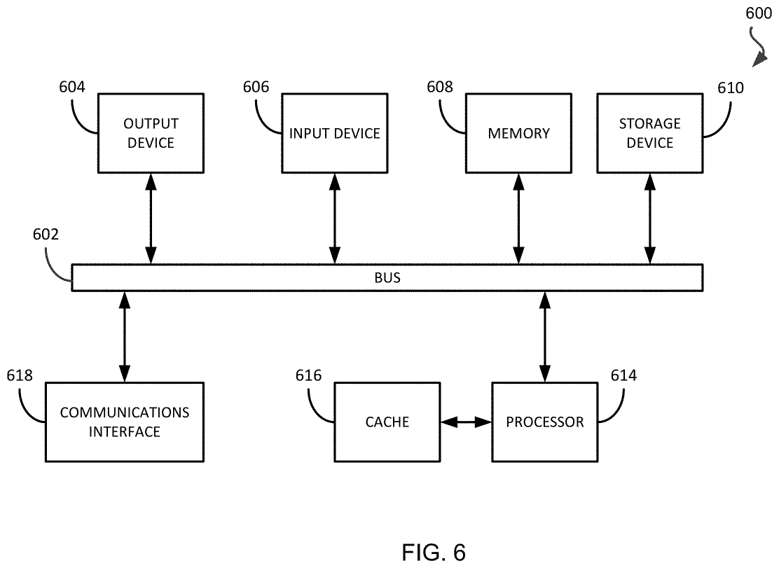

FIG. 6 depicts an example computer system 600 that may implement various systems and methods discussed herein. The computer system 600 includes one or more computing components in communication via a bus 602. In one implementation, the computer system 600 includes one or more processors 614. Each processor 614 may include one or more internal levels of cache 66, as well as bus controller or bus interface unit to direct interaction with a bus 602.

A memory 608 may include one or more memory cards and control circuits (not depicted), or other forms of removable memory, and may store various software applications including computer executable instructions, that when run on the processor 614, implement the methods and systems set out herein. Other forms of memory, such as a mass storage device 610, may also be included and accessible, by the processor (or processors) 614 via the bus 602.

The computer system 600 may further include a communications interface 618 by way of which the computer system 600 can connect to networks and receive data useful in executing the methods and system set out herein as well as transmitting information to other devices. The computer system 600 may include an output device 604, such as graphics card or other display interface by which information can be displayed on a computer monitor. The computer system 600 can also include an input device 606 by which information is input. Input device 606 can be a mouse, keyboard, scanner, and/or other input devices as will be apparent to a person of ordinary skill in the art.

The system set forth in FIG. 6 is but one possible example of a computer system that may employ or be configured in accordance with aspects of the present disclosure. It will be appreciated that other non-transitory tangible computer-readable storage media storing computer-executable instructions for implementing the presently disclosed technology on a computing system may be utilized.

The described disclosure may be provided as a computer program product, or software, that may include a computer-readable storage medium having stored thereon instructions, which may be used to program a computer system (or other electronic devices) to perform a process according to the present disclosure. A computer-readable storage medium includes any mechanism for storing information in a form (e.g., software, processing application) readable by a computer. The computer-readable storage medium may include, but is not limited to, optical storage medium (e.g., CD-ROM), magneto-optical storage medium, read only memory (ROM), random access memory (RAM), erasable programmable memory (e.g., EPROM and EEPROM), flash memory, or other types of medium suitable for storing electronic instructions.

The description above includes example systems, methods, techniques, instruction sequences, and/or computer program products that embody techniques of the present disclosure. However, it is understood that the described disclosure may be practiced without these specific details.

While the present disclosure has been described with references to various implementations, it will be understood that these implementations are illustrative and that the scope of the disclosure is not limited to them. Many variations, modifications, additions, and improvements are possible. More generally, implementations in accordance with the present disclosure have been described in the context of particular implementations. Functionality may be separated or combined in blocks differently in various embodiments of the disclosure or described with different terminology. These and other variations, modifications, additions, and improvements may fall within the scope of the disclosure as defined in the claims that follow.

We claim:

1. A computer-implemented method for mental health (MH) risk prediction comprising:
receiving mental health risk input signals for a subject, the mental health risk input signals including:
glucometer data for the subject including at least one blood glucose value, and
demographic data for the subject;
inputting the mental health risk input signals into a machine learning (ML) system previously trained with mental health risk input signals for a plurality of subjects and mental health status data for the plurality of subjects; and
obtaining a prediction of mental health risk for the subject from the ML system.

2. The computer-implemented method of claim 1, wherein the ML system comprises a neural network.

3. The computer-implemented method of claim 1, wherein the glucometer data includes one or more of:
a total number of blood glucose checks performed within a particular time interval;
a proportion of days with blood glucose checks;
a minimum blood glucose value;
a mean blood glucose;
a maximum blood glucose value;
a standard deviation of blood glucose values;
a proportion of blood glucose values below 70 mg/dl;
proportion of blood glucose values above 180 mg/dl;
a proportion of blood glucose checks with wellness indicated;
a proportion of blood glucose checks with unwell state indicated;
a proportion of blood glucose checks without a reported feeling tag; and
a proportion of blood glucose checks with exercise indicated.

4. The computer-implemented method of claim 1, wherein the demographic data includes one or more of:
age;
body mass index (BMI);
gender;
race;
diabetes status; and
smoking status.

5. The computer-implemented method of claim 1, wherein the mental health status data includes one or more of:
mental health medications prescribed for one or more of the plurality of subjects;
mental health assessments made for one or more of the plurality of subjects;
mental health insurance claims reported for one or more of the plurality of subjects; and
mental health interventions provided for one or more of the plurality of subjects.

6. The computer-implemented method of claim 1, further comprising the initial steps of:
for each subject of a first set of subjects, collecting mental health risk input signals including:
glucometer data including a glucose value,
demographic data, and
mental health status data;
creating a training set comprising the glucometer data, demographic data, and mental health status data for each subject of the first set of subjects; and
training the ML system in a training stage using the training set to create an ML model.

7. The computer-implemented method of claim 6, further comprising:
for each subject of a second set of subjects, collecting mental health risk input signals including:
glucometer data including a glucose value,
demographic data, and
mental health status data;
creating a validation set comprising the glucometer data, demographic data, and mental health status data for each subject of the second set of subjects;
validating the ML model in a validation stage using the validation set; and
updating the ML model in response to one or more validation errors.

8. The computer-implemented method of claim 1, wherein the mental health risk input signals further include coaching data relating to contacts between the subject and a coach.

9. The computer-implemented method of claim 8, wherein the coaching data includes one or more of:
a number of coaching alerts triggered;
a number of successful coach-subject contacts;
a number of successful coach-subject contacts by phone;
a number of attempted unsuccessful coach-subject contacts by phone;
a number of successful coach-subject contacts by text;
a number of attempted coach-subject unsuccessful contacts by text;
a number of successful coach-subject contacts by email;
a number of attempted unsuccessful coach-subject contacts by email;
a number of successful coach-subject contacts by glucometer;
a number of attempted unsuccessful contacts by glucometer;
a number of coaching sessions where subjects took scheduled a future coaching session; and
average minutes spent on a coaching alert interaction.

10. The computer-implemented method of claim 1, wherein the mental health risk input signals further include event data relating to one or more of frequency, duration, interactivity, and consistency of interaction sessions associated with use by the subject of a mobile application or web portal.

11. A system for mental health risk prediction comprising:
an input device for receiving mental health risk input signals for a subject, the mental health risk input signals including:
glucometer data for the subject including at least one blood glucose value, and
demographic data for the subject;
machine learning (ML) system for receiving the mental health risk input signals, wherein the ML system is previously trained with mental health risk input signals for plurality of subjects and mental health status data for the plurality of subjects; and
an output device for providing a prediction of mental health risk for the subject output by the ML system.

12. The system of claim 11, wherein the ML system comprises a neural network.

13. The system of claim 11, wherein the glucometer data includes one or more of:
a total number of blood glucose checks performed within a particular time interval;
a proportion of days with blood glucose checks;
a minimum blood glucose value;
a mean blood glucose;
a maximum blood glucose value;
a standard deviation of blood glucose values;
a proportion of blood glucose values below 70 mg/dl;
proportion of blood glucose values above 180 mg/dl;
a proportion of blood glucose checks with wellness indicated;
a proportion of blood glucose checks with unwell state indicated;
a proportion of blood glucose checks without a reported feeling tag; and
a proportion of blood glucose checks with exercise indicated.

14. The system of claim 11, wherein the demographic data includes one or more of:
age;
body mass index (BMI);
gender;
race;
diabetes status; and
smoking status.

15. The system of claim 11, wherein the mental health status data includes one or more of:
mental health medications prescribed for one or more of the plurality of subjects;
mental health assessments made for one or more of the plurality of subjects;
mental health insurance claims reported for one or more of the plurality of subjects; and
mental health interventions provided for one or more of the plurality of subjects.

16. The system of claim 11, wherein the input device, for each subject of a first set of subjects, collects mental health risk input signals including:
glucometer data including a glucose value,
demographic data, and
mental health status data; and
wherein the ML system initially receives a training set comprising the glucometer data, demographic data, and mental health status data for each subject of the first set of subjects to create an ML model.

17. The system of claim 16, wherein the input device, for each subject of a second set of subjects, collects mental health risk input signals including:
glucometer data including a glucose value;
demographic data; and
mental health status data; and
wherein the ML system validates, during a validation stage, the ML model using a validation set comprising the glucometer data, demographic data, and mental health status data for each subject of the second set of subjects, and updates the ML model.

18. The system of claim 11, wherein the mental health risk input signals further include coaching data relating to contacts between the subject and a coach.

19. The system of claim 18, wherein the coaching data includes one or more of:
a number of coaching alerts triggered;
a number of successful coach-subject contacts;
a number of successful coach-subject contacts by phone;
a number of attempted unsuccessful coach-subject contacts by phone;
a number of successful coach-subject contacts by text;
a number of attempted coach-subject unsuccessful contacts by text;
a number of successful coach-subject contacts by email;
a number of attempted unsuccessful coach-subject contacts by email;
a number of successful coach-subject contacts by glucometer;
a number of attempted unsuccessful contacts by glucometer;
a number of coaching sessions where subjects took scheduled a future coaching session; and
average minutes spent on a coaching alert interaction.

20. The system of claim 11, wherein the mental health risk input signals further include event data relating to one or more of frequency, duration, interactivity, and consistency of interaction sessions associated with use by the subject of a mobile application or web portal.

21. A computer-readable medium including program instructions that, when executed by a processor, cause the processor to perform a method for mental health (mental health) risk prediction, the method comprising:
receiving mental health risk input signals for a subject, the mental health risk input signals including:
glucometer data for the subject including at least one blood glucose value, and
demographic data for the subject;
inputting the mental health risk input signals into a machine learning (ML) system previously trained with mental health risk input signals for plurality of subjects and mental health status data for the plurality of subjects; and
obtaining a prediction of mental health risk for the subject from the ML system.
